(19) United States
(12) Patent Application Publication
(10) Pub. No.: US 2015/0005530 A1
(43) Pub. Date: Jan. 1, 2015

(54) L-ENANTIOMERS SELECTIVE MEMBRANE FOR OPTICAL RESOLUTION OF ALPHA-AMINO ACIDS AND PROCESS FOR THE PREPARATION THEREOF

(71) Applicant: Council Scientific & Industrial Research, New Delhi (JP)

(72) Inventors: Kripal Singh, Gujarat (IN); Hari Chand Bajaj, Gujarat (IN); Pravin Ganeshrao Ingole, Gujarat (IN)

(21) Appl. No.: 14/377,005

(22) PCT Filed: Feb. 6, 2013
(86) PCT No.: PCT/IN2013/000081

§ 371 Date: Aug. 6, 2014

(30) Foreign Application Priority Data
Feb. 6, 2012 (IN) 0327/DEL/2012

(51) Int. Cl. B01D 71/56; C07C 227/34; B01D 61/14; B01D 69/12; B01D 67/00

(52) U.S. Cl. CPC B01D 71/56; B01D 69/125; B01D 67/0006; B01D 61/145; C07C 227/34; B01D 2323/30; B01D 2323/40; B01D 2323/36; B01D 2325/04
USPC 562/402; 210/500.38; 427/245

(57) ABSTRACT

The present invention provides a L-enantiomers selective composite membrane useful for separation of optical isomers and the process for the preparation thereof. The invention further provides a membrane based pressure driven separation process for separation of enantiomers from their mixture to obtain optical pure isomers. The present invention also provides a membrane based method for optical resolution of racemic mixtures of amino acids to obtain optically pure amino acids.

The following specification particularly describes the invention and the manner in which it is to be performed:

### FIELD OF THE INVENTION

The present invention relates to L-enantiomers selective membrane for optical resolution of racemic mixtures of α-amino acids. Particularly, present invention relates to a method of preparation of enantioselective composite nanofiltration membrane useful for separation of optical isomers of α-amino acids. More particularly, present invention relates to enantioselective composite membrane, useful for optical resolution of racemic mixtures of α-amino acids and chiral compounds to obtain optically pure enantiomers through pressure driven membrane process.

### BACKGROUND OF THE INVENTION

Stereoisomers are those molecules that differ from each other only in the arrangement of their atoms within space. Stereo-isomers are generally classified as diastereomers or enantiomers; the latter embracing those which are mirror images of each other and former being those which are not mirror images. Enantiomers (the mirror images), also known as optical isomers, have identical physical and chemical properties. Therefore a mixture of enantiomers as a rule can not be separated by ordinary separation methods, such as fractional distillation (boiling points being identical), as conventional crystallization unless the solvent is optically active (due to identical solubilities), as conventional chromatography unless adsorbent is optically active (because they are held equally onto ordinary adsorbent). The problem of separating enantiomers is further exacerbated by the fact that conventional synthetic techniques usually produce a mixture of enantiomers. Thus, separation of a mixture of enantiomers is a most challenging problem in analytical chemistry. Separation of enantiomers is very important to organic compounds such as amino acids, drugs, pesticides, insecticides etc. because majority are optically active and exist as a pairs of optical isomers (enantiomers). Enantiomers of many chiral drugs show remarkably differences in their biological a pharmacological properties. One enantiomer may have drug activity, while the other may be inert or even harmful. For examples, (S)-verapamil is effective as a calcium channel blocker while (R)-verapamil produces cardiac side effects; L-enantiomer of B-blocker propranolol is ˜100 times more active than L-form; (R)(+)-enantiomer of thalidomide possesses the sleeping action and its (S)(−)-enantiomer possesses teratogenic action, the different in pharmacological action of thalidomide was found responsible for serious malformation in newborn babies of women who took drug during pregnancy, “Thalidomide Tragedy” in 1960's etc. It is therefore “The United States Food and Drug Administration” has recently issued new regulations governing the marketing of chiral drugs. According to the new regulations, the pharmacological properties of each enantiomer of a chiral drug should be tested separately for therapeutic efficacy and safety.

Various methods are known for separating enantiomers such as diastereomeric resolutions, enzyme catalyzed reactions, chromatographic methods, the application of liquid membranes, molecular recognition techniques, and inclusion complexation techniques. Preferential crystallization, diastereomeric resolutions, enzyme catalyzed reactions etc. involve coupling of the enantiomers with an auxiliary chiral reagent to convert them into diastereomers, which can then be separated by any conventional separation technique. Reference may be made to Diastereomeric resolutions and are described in “CRC Handbook of Optical Resolutions via Diasteromeric Salt Formation” Kozma D., 2002 ISBN: 0849300193. The major drawback of diastereomeric resolutions is the requirement of large quantities of optically pure derivatizing agent (chiral reagents or solvents) which can be expensive and can often not be recoverable.

References may be made to Chromatographic techniques (GC, HPLC, CE, SFC, etc.) and are described in “Chiral Separation Techniques—A Practical Approach” Second Edition, Edited by G. Subramanian ISBN 3-527-29875-4, wherein Chromatographic methods require an appropriate chiral selector incorporated into the stationary phase (chiral stationary phase) or coated onto the surface of the column packing material (chiral coated stationary phases). Enantioselective Chiral columns having chiral stationary phases are costly and have finite working life. Therefore cost of separation is quite high.

References may be made to Molecular recognition phenomena for enantiomers separation has been reported in “Chiral Separation Techniques—A Practical Approach” Second Edition, Edited by G. Subramanian ISBN 3-527-29875-4. A varying nos. of chiral stationary phases, complexes etc. have been developed based of molecular recognition.

References may be made to U.S. Pat. No. 6,485,650 entitled “Liquid membrane separation of enantiomers”, wherein a method of separating enantiomers in a supported liquid membrane module containing a carrier and a phase transfer agent with a feed fluid containing a racemic mixture describes an enantiomer which is transported into the liquid membrane and thereafter contacting the liquid membrane with a sweep fluid. The enantiomer is then recovered from the sweep fluid. The membrane module is constructed in such a way that the feed fluid and the sweep fluid are adjacent to, but on opposite sides of, the liquid membrane and the feed and sweep fluids have a substantially continuous interfacial contact along the length of the liquid membrane. The drawbacks of the liquid-liquid extraction technique are less productivity and chances of inter-mixing the two solutions at the interface of the membrane. References may be made to U.S. Pat. No. 5,080,795 entitled “Supported chiral liquid membrane for the separation of enantiomers”, wherein supported chiral liquid membrane containing chiral carrier selectively complexes with one of the two enantiomers and separates it from other. The major drawbacks of the membrane are poor stability and loss of enantioselectivity with time.

References may be made to U.S. Pat. No. 6,013,738 entitled “Composition and method for chiral separations” wherein polymers are disclosed for the chiral separations, these polymers are novel polymers but main drawbacks is the synthesis route is very long.

References may be made to U.S. Pat. No. 6,265,615 entitled “Chiral recognition polymer and its use to separate enantiomers” wherein a polymer film or powder and the process using these materials are used to perform enantiomers separation of amino acids and pharmaceuticals compounds. This dedoped film results in lessening of the separation capability over a period of time. The membrane can be formed from polyaniline doped with a chiral acid and then extracted with a suitable base; preferably one enantiomer is released after contacting racemic mixture with the surface of film. Drawback of this method is the enantioselectivity is comparatively less than other methods.

References may be made to U.S. Pat. No. 4,277,344 entitled “Interfacially synthesized reverse osmosis membrane”, wherein an aromatic polyamide film which is the interfacial reaction product of an aromatic polyamine having at least two primary amines groups with an aromatic acyl halide having at least three acyl halide groups. According to this patent a porous polysulfone support is coated with m-phenylenediamine in water. After removal of excess m-phenylenediamine solution from the coated support, the coated support is covered with a solution of trimesoyl chloride dissolved in “FREON” TF solvent (trichlorotrifluoroethane). The contact time for the interfacial reaction is 10 seconds, and the reaction is substantially complete in 1 second. The resulting polysulfone/polyamide composite membrane is then air-dried. The membrane claims to exhibits good flux and salt rejection. However, in order to improve the membrane performance various types of additives have been incorporated into the solutions used in the interfacial polycondensation reaction. The drawback of this membrane is that it is not enantioselective.

References may be made to U.S. Pat. No. 5,205,934 entitled “Silicone-derived solvent stable membranes” wherein methods for producing composite nanofiltration membranes describe, which comprise a layer of silicone immobilized onto a support, preferably a polyacrylonitrile support. These composite membranes are claimed to be solvent stable and are claimed to have utility for separation of high molecular weight solutes, including organometallic catalyst complexes, from organic solvents. The membrane does not have enantioselective character.

References may be made to Journal Indian Journal of Chemical Technology Vol. 18, May 2011 entitled “Optical resolution of alpha amino acid derivative through membrane process” describes a D-enantiomers selective membrane prepared by interfacial technique using L-lysine, piperazin and trimesoyl chloride as reactive monomers. The membrane rejects lysine more than 60% with ˜95% ee.

The enantioselective polymer membranes described in prior arts as detailed above are asymmetric and dense membranes fabricated from chiral polymers such as polysaccharides and derivatives, poly α-amino acids, polyacetylene derivatives etc. Most of these polymers are crystalline in nature and do not have membrane forming ability. Therefore membranes made from such polymers are fragile hence difficult to handle. Poor mechanical properties restricted their use to dialysis mode of separation. In dialysis mode of separation the driving force is solute concentration gradient only, therefore these membranes exhibited very low rate of permeation. Other types of enantiomers separation membranes are prepared from non chiral polymers having grafted enantiomers recognizing molecules viz.; amino acids, proteins, oligo-peptides etc. These membranes have superior mechanical properties however during permeation recognition sites get saturated quickly being fixed in the polymer matrix therefore selectivity of such membranes decrease sharply with time.

Composite membranes are typically prepared by coating a porous support membrane with an aqueous solution of polyfunctional amine, followed by coating with solution of a polyfunctional acyl halide in an organic solvent to prepare thin film discriminating layer of polyamide by interfacial polycondensation reaction between a polyfunctional amine and a polyfunctional acyl halide as described in various patents.

The inventive steps involved in the present invention are i) top discriminating layer of composite membrane has resulted by interfacial polymerization reaction of chiral amino acids and polyfunctional amine with polyfunctional acyl chloride , (ii) the preparation of top chiral enantioselective layer by interfacial method requires very small amount of chiral compound and very large membrane having homo chiral environment can be fabricated, (iii) the process minimizes the requirement of optically pure chiral reagent essential for the separation of racemic mixtures, and (iv) the process bring chiral micro environment in the polymer membrane in the form of top thin layer supported on the ultrafiltration layer which results in higher flux and high selectivity.

### OBJECTIVE OF THE INVENTION

The main object of the present invention is to provide a L-enantioselective composite membrane comprising ultrafiltration membrane having thickness in the range of 20-60 μm coated with cross linked polyamide polymer having thickness in the range of 500 to 1600 Å wherein said polymer contain at least one chiral carbon atom.

Another object of the present invention is to provide a method for the preparation of enantioselective composite membrane that obviates the drawbacks as detailed above.

Another object of the present invention is to provide a method for the fabrication of a self-supporting and perm-selective membrane for enantiomeric separation through pressure driven membrane process.

Still another object of the present invention is to provide a method for fabricating enantioselective composite nanofiltration membrane for separation of enantiomers of chiral molecules.

Yet another object of the present invention is to provide a membrane based separation method for optical resolution of a racemic mixture into optically pure isomers.

Yet another object of the present invention is to provide a method to obtain optically pure isomers of amino acids.

### BRIEF DESCRIPTION OF DRAWING

FIG. 1: FIG. 1 shows Attenuated total reflectance-Fourier transform infrared spectroscopy (ATR-FTIR) spectra of

A1-polysulfone(PS),

B1 (2% trans 1,4-diamino cyclohexane: 1% TMC (Trimesoyl chloride) composite membrane.

B2 (4% trans 1,4-diamino cyclohexane: 1% TMC) composite membrane.

B3 (6% trans 1,4-diamino cyclohexane: 1% TMC) composite membrane.

FIG. 2: Scanning Electron Microscopy (SEM) Analysis

a) Surface view of modified membrane b) Cross sectional view of modified membrane.

FIG. 3: Atomic Force Microscopy (AFM) Analysis

2D-AFM images of composite membrane (a), 3D-AFM images of composite membrane (b).

### SUMMARY OF THE INVENTION

Accordingly, present invention provides L-enantioselective composite membrane comprising ultrafiltration membrane having thickness in the range of 20-60 μm coated with cross linked polyamide polymer having thickness in the range of 500 to 1600 Å wherein said polymer contain at least one chiral carbon atom.

In an embodiment of the present invention, ultrafiltration membrane used is selected from the group consisting of polysulfone, polyethersulfone, and polyvinylidienefluoride.

In an embodiment, present invention provides a method for preparation of L-enantioselective composite membrane as claimed in claim 1 and the said process comprising the steps of:
i. providing ultrafiltration (UF) membrane prepared by wet phase inversion method;
ii. mixing polyfunctional amine and acid acceptor to obtain 2-6% aqueous solution;
iii. dip coating of ultrafiltration membrane as provided in step (i) in solution as obtained in step (ii) for a period in the range of 1 to 5 minutes maintaining the pH in the range of 10 to 13 followed by removing and draining the extra solution from the UF membrane for a period in the range of 5 to 20 minutes to obtain coated membrane;
iv. again dipping the coated membrane as obtained in step (iii) in 1-2% solution of triacyl halide in hexane for a period in the range of 1 to 5 minutes followed by draining the extra solution for a period in the range of 1 to 5 minutes;
v. drying the membrane as obtained in step (iv) for a period in the range of 1 to 2 hours;
vi. heating the membrane as obtained in step (v) for a period in the range of 5 to 15 minutes at a temperature in the range of 70° C. to 90° C. followed by cooling and air drying for a period in the range of 1 to 2 hours;
vii. soaking the membrane as obtained in step (vi) in deionized water up to 24 hours to obtain L-enantioselective composite membrane.

In another embodiment of the present invention, the ultrafiltration membrane used is selected from the group consisting of polysulfone, polyethersulfone, and polyvinylidienefluoride having thickness in the range of 20-60 μm.

In yet another embodiment of the present invention, acid acceptor used is selected from triethyl amine or NaOH, preferably NaOH.

In yet another embodiment of the present invention, polyfunctional amine used is used is selected from the group consisting of at least two primary amino groups preferably trans 1,4-diamino cyclohexane.

In yet another embodiment of the present invention, triacyl halide used is trimesoyl chloride.

In yet another embodiment of the present invention, enantioselective composite membrane separate enantiomers up to 75-97% ee arginine, 76-95% ee lysine, 76-91% ee cystein and 52-81% ee asparagine from aqueous solution of respective racemic amino acids.

In yet another embodiment, present invention provides a method for enantio-separation of racemic mixture of α-amino acids, using the enantioselective composite membrane, obtained by the process as claimed in claim 1, wherein the said process is carried out on a reverse osmosis membrane testing unit at trans membrane pressure ranging between 50 psi to 150 psi, using aqueous and/or buffered solution of amino acids in the range of 0.1 to 1% as feed at flow rate in the range of 300 to 800 ml per minute at 20-30° C.

In yet another embodiment of the present invention, concentration of amino acids in permeate was determined by UV-Vis spectrophotometer and the ratio of D and L-enantiomers in permeate was estimated on HPLC fitted with PDA detector, by using Chiral column.

### DETAILED DESCRIPTION OF THE INVENTION

Enantioselective thin film composite membranes of the present invention are prepared by coating a micro-porous support with trans 1,4-diamino cyclohexane (having two primary amino groups) and an acid acceptor triethyl amine, NaOH preferably NaOH and then a polyfunctional acyl halide (having reactivity more than one) preferably trimesoyl chloride stepwise. The coating steps need not be in specific order; however trans 1,4-diamino cyclohexane and acid acceptor is preferably coated first followed by coating of polyfunctional acyl halide. The trans 1,4-diamino cyclohexane is coated from an aqueous solution and polyfunctional acyl halide is coated from an organic solution.

First ultrafiltration membrane is fabricated from polymer materials such as Polysulfone, Polyethersulfone, Polyvinylidieneflouride, etc. preferably polysulfone by phase inversion technique. In this technique, a solution of above-mentioned polymers of desired concentration 12 to 18% w/w in aprotic solvents such as dimethylformamide, N, N dimethylacetamide etc (more precisely 18% w/w) is spreaded on non-woven polyester fabric (support) in uniform thickness, the support is then dipped in coagulation bath containing 2% aqueous solution of dimethylformamide after specified time varies from 10-40 seconds. The membrane is washed with deionised water for several times.

Ultrafiltration membrane so prepared is used for the preparation of enantioselective composite membranes of present invention, by preparing a thin enantioselective layer in-situ on the top of ultrafiltration membrane by interfacial polymerization technique by reacting 2-6% aqueous solution of a trans 1,4-diamino cyclohexane and an acid acceptor viz., triethyl amine, NaOH etc., preferably NaOH. The pH of aqueous solution is maintained at 10-13 preferably 12, with 1-2% solution of trimesoyl chloride in hexane.

To prepare enantioselective layer on the top of ultrafiltration membrane it is first dip coated with aqueous solution of trans 1,4-diamino cyclohexane and an acid acceptor viz., triethyl amine, NaOH etc. for 1-5 minutes precisely 3 minutes. The coated UF membrane is removed from the solution and excess solution is drained from UF membrane for about 5-20 minutes precisely 15 minutes to retain the desired amount of monomer/monomers.

The UF membrane is then dip coated with 1-2% solution of trimesoyl chloride in hexane precisely 1.0%, for a period of about 1-5 minutes precisely 3 minutes. The resultant coated UF membrane is removed from trimesoyl chloride solution mixture and membrane is drained off for 1-5 minutes precisely for 5 minutes to remove excess solution of trimesoyl chloride. The membrane is then air dried for 1-2 h precisely 2 h, then cured by heating at a temperature of 70-90° C. precisely at 80° C. for 5-15 minutes, precisely for 10 minutes. The resultant membrane is then cooled and dried in air for two hours and then soaked in water up to 24 hours to obtain the desired enantioselective composite membrane.

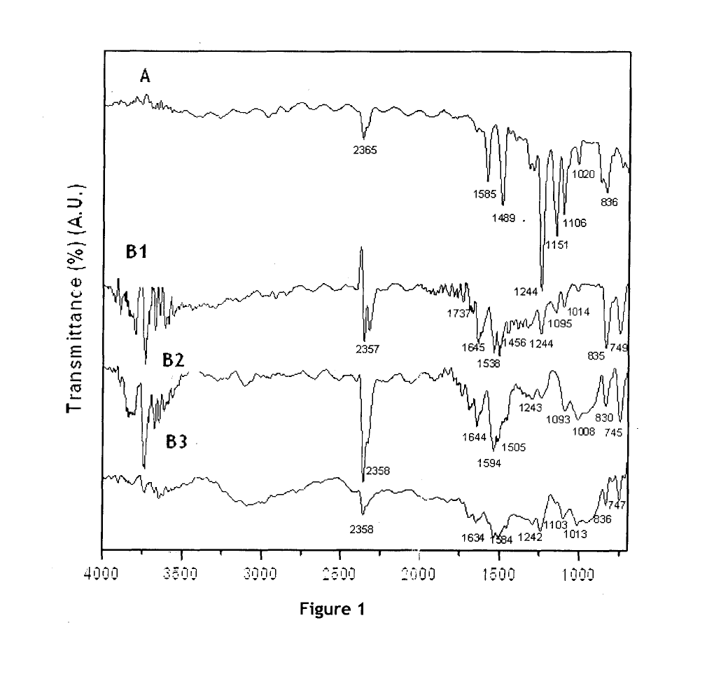

FIG. 1: The enantioselective composite membrane was characterized by ATR-FTIR spectrophotometer for chemical structure of its top layer. ATR-FTIR spectra of polysulfone membrane before coating and after coating were recorded on a Perkin-Elmer spectrometer (Perkin-Elmer Spectrum GX, ATR-FTIR) using a Germanium crystal at a nominal incident angle of 45° at speed of 100 scans at a resolution of 2 cm⁻¹. ATR-FTIR spectra of polysulfone membrane (A) and after coating (B1, B2, B3) it with poly (piperazinecoarginine trimesamide) film in-situ are given in FIG. 1. The peaks corresponding polysulfone were observed at 1484-1490 cm⁻¹ and 1587 cm⁻¹. The appearance of absorption bands in 1475-1650 cm⁻¹ region may be related to the C═O, C═N groups. The peak arises at 1644-1710 cm⁻¹ in coated membrane is due to amide linkage. The characteristic absorption bands at 1720 cm⁻¹ (imide ring C═O), 1680 cm⁻¹ (imine group), 1372 cm⁻¹ (C—N—C, imide in the plane) and at 739 cm⁻¹ (C—N—C, out-of-plane bending, imide) observed in composite membranes.

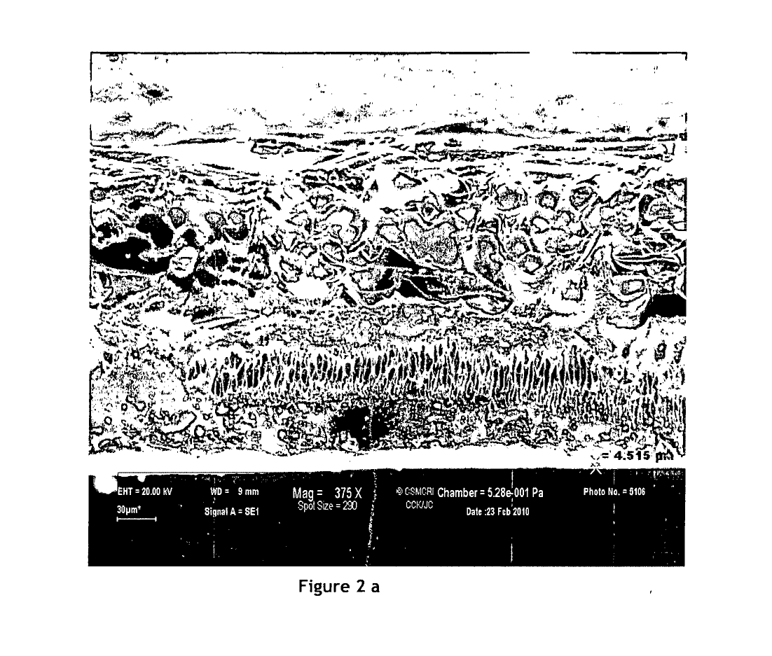

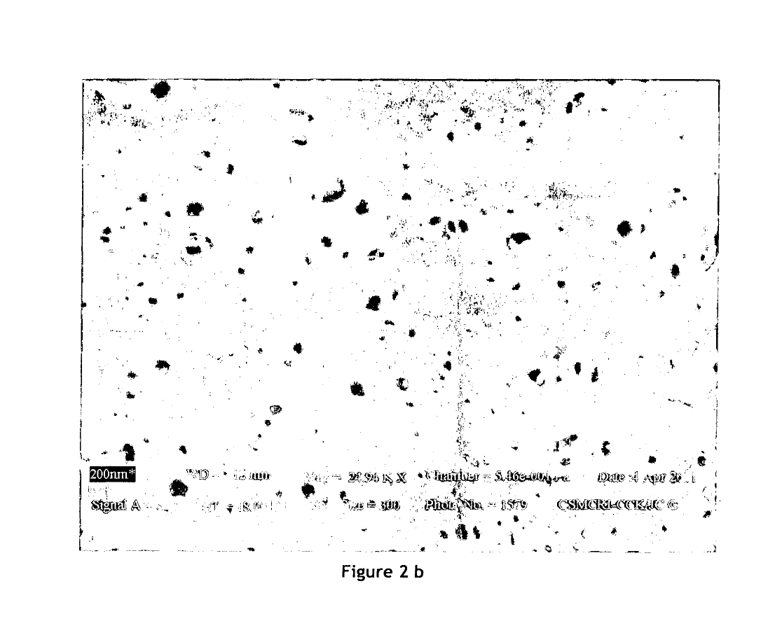

FIG. 2: The enantioselective composite membrane was characterized by Scanning Electron Microscopy (SEM) using Leo, 1430UP, Oxford instruments. The surface morphology of membranes is examined through scanning electron microscope (surface view and cross section) given in FIG. 2 clearly shows three layers in the membrane correspondence to non-woven polyester fabric, micro porous polysulfone layer and enantioselective polymer layer.

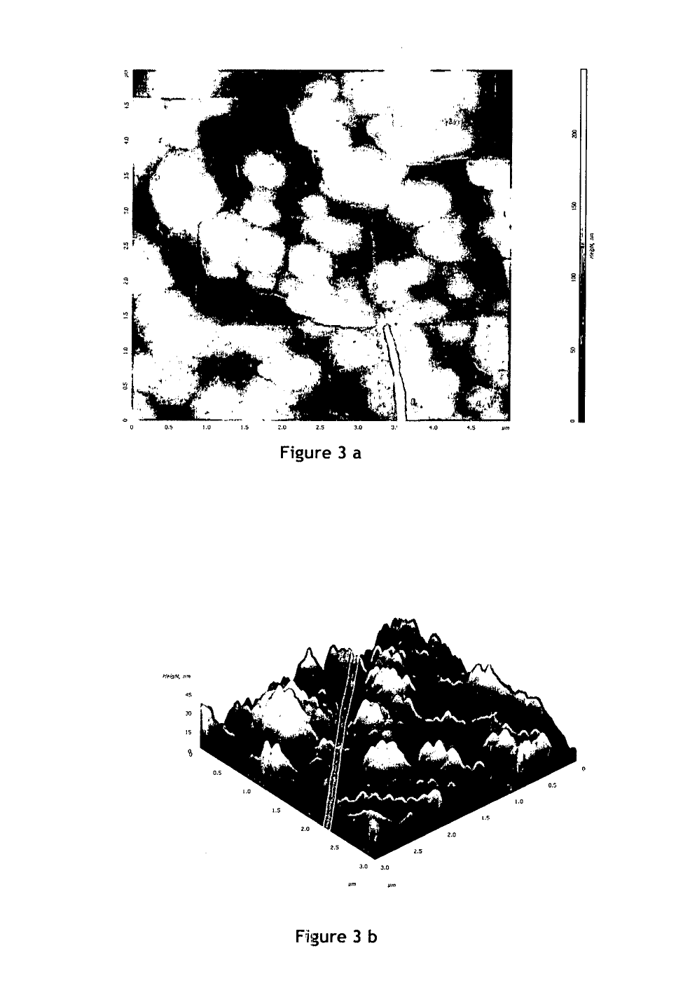

FIG. 3: The enantioselective composite membrane was characterized by Atomic Force Microscopy (AFM). AFM images of membranes were taken on an AFM/SPM instrument (Ntegra Aura Model NT-MDT-MOSCOW) in semi contact mode. AFM images shows morphology of PS and composite membranes. The surface of membranes indicates a typical nodular (hills and valleys) morphology inherent to the surfaces prepared by interfacial polymerization. The images of composite membranes showed some less roughness compared to the PS membrane.

The membrane was tested for separation of α-amino acids (arginine, lysine. cystein, and asparagine) from their aqueous and buffered solutions through reverse osmosis at trans-membrane pressure in the range of 50-150 psi, precisely at 75 psi, using 0.1-1%, aqueous solution and buffer solution of α-amino acids as feed at flow rate varies from 300-800 ml per minute precisely 500 ml per minute at ambient temperature. The concentration of amino acids in permeate was determined by UV-Vis spectrophotomer at 290 nm and the ratio of D and L-enantiomers in permeate to determine the enantiomeric excess (ee %) was estimated on HPLC fitted with PDA detector, by using Chiral column Chrompak (+) supplied by Diacel Chemical Industries, USA.

Enantiomers are chiral molecules having identical molecular formula and chemical structure, but differ only in their spatial orientation. The difference in spatial orientation has many implications as biological and pharmaceutical activities of many chiral compounds are entirely different. Therefore, use of such compounds in optically pure form is imminent. The separation of enantiomers presents a difficult problem. Many techniques are known in the art for separation of enantiomers based on different techniques. All enantioseparation techniques are based on the presence of chiral microenvironment in the separation process for identifying the paired enantiomers.

The presence of homo-chiral environment is essential to discriminate paired enantiomers. The novelty of the membrane of the present invention is to bring chiral micro environment in the polymer membrane in the form of top thin layer supported on the ultrafiltration layer which results higher flux and higher selectivity.

The composite membranes of present invention have enantioselective top layer chiral discriminating layer that has been prepared in-situ on the top of ultrfiltration. Top discriminating layer has resulted by interfacial polymerization reaction of chiral amino acids and polyfunctional amine with polyfunctional acyl chloride. The Preparation of top chiral enantioselective layer by interfacial method requires very small amount of chiral compound and very large membrane having homo-chiral environment can be fabricated. Thus minimizes the requirement of optically pure chiral reagent essential for separation of racemic mixtures.

### EXAMPLES

The following examples are given by way of illustration and therefore should not be construed to limit the scope of the present invention.

### Example 1

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for arginine at standard conditions; 0.1% aqueous solution of racemic arginine as feed. Membrane exhibited permeation rate 48 gfd and 94% enantioselectivity for L-arginine was observed.

### Example 2

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for arginine at standard conditions; 0.1% aqueous solution of racemic arginine as feed. Membrane exhibited permeation rate 42 gfd and 75% enantioselectivity for L-arginine was observed.

### Example 3

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for arginine at standard conditions; 0.1% aqueous solution of racemic arginine as feed. Membrane exhibited permeation rate 36 gfd and 97% enantioselectivity for L-arginine was observed.

### Example 4

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for arginine at standard conditions; 0.1% aqueous solution of racemic arginine as feed. Membrane exhibited permeation rate 32 gfd and 85% enantioselectivity for L-arginine was observed.

### Example 5

Enantioselective composite membrane was prepared by impregnating polysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for arginine at standard conditions; 0.1% aqueous solution of racemic arginine as feed. Membrane exhibited permeation rate 32 gfd and 94% enantioselectivity for L-arginine was observed.

### Example 6

Enantioselective composite membrane was prepared by impregnating polysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then o drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for arginine at standard conditions; 0.1% aqueous solution of racemic arginine as feed. Membrane exhibited is permeation rate 30 gfd and 81% enantioselectivity for L-arginine was observed.

### Example 7

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for lysine at standard conditions; 0.1% aqueous solution of racemic lysine as feed. Membrane exhibited permeation rate 42 gfd and 95% enantioselectivity for L-lysine was observed.

### Example 8

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for lysine at standard conditions; 0.1% aqueous solution of racemic lysine as feed. Membrane exhibited permeation rate 33 gfd and 85% enantioselectivity for L-lysine was observed.

### Example 9

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl is chloride in hexane for 2 minutes, extra solution was drained for 2 minutes then drying the membrane for 2 hours in air. The membrane was, heat cured for 10 minutes at 80° C. temperature, cooled to ambient temperature; air dried for 2 hours, and then soaked in deionized water up to 24 hours. The membrane was tested for separation and enantioselectivity for lysine at standard conditions; 0.1% aqueous solution of racemic lysine as feed. Membrane exhibited permeation rate 42 gfd and 93% enantioselectivity for L-lysine was observed.

### Example 10

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for lysine at standard conditions; 0.1% aqueous solution of racemic lysine as feed. Membrane exhibited permeation rate 40 gfd and 92% enantioselectivity for L-lysine was observed.

### Example 11

Enantioselective composite membrane was prepared by impregnating polysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then to drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for lysine at standard conditions; 0.1% aqueous solution of racemic lysine as feed. Membrane exhibited permeation rate 37 gfd and 81% enantioselectivity for L-lysine was observed.

### Example 12

Enantioselective composite membrane was prepared by impregnating polysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for lysine at standard conditions; 0.1% aqueous solution of racemic lysine as feed. Membrane exhibited permeation rate 31 gfd and 76% enantioselectivity for L-lysine was observed.

### Example 13

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for cystein at standard conditions; 0.1% aqueous solution of racemic cystein as feed. Membrane exhibited permeation rate 50 gfd and 91% enantioselectivity for L-cystein was observed.

### Example 14

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl is chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for cystein at standard conditions; 0.1% aqueous solution of racemic cystein as feed. Membrane exhibited permeation rate 46 gfd and 90% enantioselectivity for L-cystein was observed.

### Example 15

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for cystein at standard conditions; 0.1% aqueous solution of racemic cystein as feed. Membrane exhibited permeation rate 48 gfd and 83% enantioselectivity for L-cystein was observed.

### Example 16

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for cystein at standard conditions; 0.1% aqueous solution of racemic cystein as feed. Membrane exhibited is permeation rate 40 gfd and 89% enantioselectivity for L-cystein was observed.

### Example 17

Enantioselective composite membrane was prepared by impregnating polysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for cystein at standard conditions; 0.1% aqueous solution of racemic cystein as feed. Membrane exhibited permeation rate 42 gfd and 85% enantioselectivity for L-cystein was observed.

### Example 18

Enantioselective composite membrane was prepared by impregnating polysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for cystein at standard conditions; 0.1% aqueous solution of racemic cystein as feed. Membrane exhibited permeation rate 36 gfd and 76% enantioselectivity for L-cystein was observed.

### Example 19

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for asparagine at standard conditions; 0.1% aqueous solution of racemic asparagine as feed. Membrane exhibited permeation rate 52 gfd and 81% enantioselectivity for L-asparagine was observed.

### Example 20

Enantioselective composite membrane was prepared by impregnating polysulfone UF membrane in 2% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for asparagine at standard conditions; 0.1% aqueous solution of racemic asparagine as feed. Membrane exhibited permeation rate 48 gfd and 76% enantioselectivity for L-asparagine was observed.

### Example 21

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for. 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for asparagine at standard conditions; 0.1% aqueous solution of racemic asparagine as feed. Membrane exhibited permeation rate 50 gfd and 71% enantioselectivity for L-asparagine was observed.

### Example 22

Enantioselective composite membrane was prepared by impregnating polysulfone UF membrane in 4% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for asparagine at standard conditions; 0.1% aqueous solution of racemic asparagine as feed. Membrane exhibited permeation rate 44 gfd and 67% enantioselectivity for L-asparagine was observed.

### Example 23

Enantioselective composite membrane was prepared by impregnating potysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 1.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for asparagine at standard conditions; 0.1% aqueous solution of racemic asparagine as feed. Membrane exhibited permeation rate 35 gfd and 57% enantioselectivity for L-asparagine was observed.

### Example 24

Enantioselective composite membrane was prepared by impregnating polysulfone UF membrane in 6% aqueous solution of trans 1,4-diamino cyclohexane for 3 minutes, pH of solution was maintained to 12 by adding 1N NaOH, draining extra solution for 15 minutes and then dipping membrane in 2.0% solution of trimesoyl chloride in hexane for 2 minutes, extra solution was drained for 2 minutes then drying the membrane for 2 hours in air. The membrane was heat cured for 10 minutes at 80° C. temperature, cooled to ambient temperature; air dried for 2 hours, and then soaked in deionized water up to 24 hours. The membrane was tested for separation and enantioselectivity for asparagine at standard conditions; 0.1% aqueous solution of racemic asparagine as feed. Membrane exhibited permeation rate 31 gfd and 52% enantioselectivity for L-asparagine was observed.

### ADVANTAGES OF THE INVENTION

1) The enantioselective polymer membranes described in prior art are asymmetric and dense membranes fabricated from chiral polymers such as polysaccharides and derivatives, trans 1,4-diamino cyclohexane, polyacetylene derivatives etc. Most membranes are fragile have poor mechanical properties thus posses difficulties to handle membrane, as a result their use is restricted to dialysis mode of separation. In dialysis mode of separation the driving force is solute concentration across the membrane therefore membranes exhibit very low rate of permeation. Membranes having superior mechanical properties exhibit enantioselectivity in the beginning but selectivity decrease sharply with time due to saturation of recognition sites.
2) The composite membranes of the present invention obviate the drawbacks of the membrane described in prior arts as mentioned above.
3) The composite membranes of the present invention can be used to perform enantiomers separation at commercial scale.
4) The composite membranes of the present invention exhibits permeation flux of 30-52 gfd depending upon trans-membrane pressure.
5) The composite membranes of present invention can be used in pressure driven separation process at pressure varies from 50 to 150 psi. The higher trans-membrane pressure result higher flux thereby higher productivity.
6) The composite membranes of present invention are stable and mechanically superior therefore it is to handle and convert into modular form.
7) The enantiomers separation methods described in prior arts are often batch processes even if continuous, could not be adapted for a large scale continuous separation. The enantiomers separation process using membranes of present invention would be a continuous process and can be adapted for a large scale continuous separation.
8) The enantiomers separation process of present invention would exhibit high rate of transport and the degree of separation in a reasonable time period to make feasible for large scale α-amino acids separation from their aqueous solution and mixture.

1. L-enantioselective composite membrane comprising ultrafiltration membrane having thickness in the range of 20-60 μm coated with cross linked polyamide polymer having thickness in the range of 500 to 1600 Å wherein said polymer contain at least one chiral carbon atom.

2. The enantioselective composite membrane as claimed in claim 1, wherein ultrafiltration membrane used is selected from the group consisting of polysulfone, polyethersulfone, and polyvinylidienefluoride.

3. The enantioselective composite membrane as claimed in claim 1, wherein enantioselective composite membrane separates enantiomers up to 75-97% ee arginine, 76-95% ee lysine, 76-91% ee cystein and 52-81% ee asparagine from aqueous solution of respective racemic amino acids.

4. A method for preparation of L-enantioselective composite membrane as claimed in claim 1 and the said process comprising the steps of:
i. providing ultrafiltration (UF) membrane prepared by wet phase inversion method;
ii. mixing polyfunctional amine and acid acceptor to obtain 2-6% aqueous solution;
iii. dip coating of ultrafiltration membrane as provided in step (i) in solution as obtained in step (ii) for a period in the range of 1 to 5 minutes maintaining the pH in the range of 10 to 13 followed by removing and draining the extra solution from the UF membrane for a period in the range of 5 to 20 minutes to obtain coated membrane;
iv. again dipping the coated membrane as obtained in step (iii) in 1-2% solution of triacyl halide in hexane for a period in the range of 1 to 5 minutes followed by draining the extra solution for a period in the range of 1 to 5 minutes;
v. drying the membrane as obtained in step (iv) for a period in the range of 1 to 2 hours;
vi. heating the membrane as obtained in step (v) for a period in the range of 5 to 15 minutes at a temperature in the range of 70° C. to 90° C. followed by cooling and air drying for a period in the range of 1 to 2 hours;
vii. soaking the membrane as obtained in step (vi) in deionized water up to 24 hours to obtain L-enantioselective composite membrane.

5. The method as claimed in claim 4, wherein the ultrafiltration membrane used in step (i) is selected from the group consisting of polysulfone, polyethersulfone, and polyvinylidienefluoride having thickness in the range of 20-60 μm.

6. The method as claimed in claim 4, wherein the acid acceptor used in step (ii) is selected from triethyl amine or NaOH, preferably NaOH.

7. The method as claimed in claim 4, wherein the polyfunctional amine used in step (ii) is selected from the group consisting of at least two primary amino groups preferably trans 1,4-diamino cyclohexane.

8. The method as claimed in claim 4, wherein triacyl halide used in step (iv) is trimesoyl chloride.

9. A method for enantio-separation of racemic mixture of α-amino acids, using the enantioselective composite membrane as claimed in claim 1, wherein the said process is carried out on a reverse osmosis membrane testing unit at trans membrane pressure ranging between 50 psi to 150 psi, using aqueous and/or buffered solution of amino acids in the range of 0.1 to 1% as feed at flow rate in the range of 300 to 800 ml per minute at 20-30° C.

10. The method as claimed in claim 9, wherein concentration of amino acids in permeate was determined by UV-Vis spectrophotometer and the ratio of D and L-enantiomers in permeate was estimated on HPLC fitted with PDA detector, by using Chiral column.
